(19) United States
(12) Patent Application Publication
(10) Pub. No.: US 2006/0034780 A1
(43) Pub. Date: Feb. 16, 2006

(54) Dental compositions and methods

(75) Inventors: Yue Hugh Guan, Sheffield (GB); Terence Henry Lilley, Sheffield (GB)

Correspondence Address: ROTHWELL, FIGG, ERNST & MANBECK, P.C., 1425 K STREET, N.W., SUITE 800, WASHINGTON, DC, 20005, US

(73) Assignees: The Boots Company, Nottingham (GB); The University of Sheffield, Sheffield, South Yorkshire (GB)

(21) Appl. No.: 10/528,828

(22) PCT Filed: Sep. 23, 2003
(86) PCT No.: PCT/GB03/04162

§ 371 Date: Mar. 23, 2005

(30) Foreign Application Priority Data
Sep. 24, 2002 (GB) 0222091.1

(51) Int. Cl. A61K 8/49; A61K 8/31

(52) U.S. Cl. 424/49

(57) ABSTRACT

A dental composition comprises a fluorescent whitening agent. The fluorescent whitening agent is most preferably 4,4′-bis(2-sulfostryryl)bipheny) or a salt or other soluble derivative thereof, eg the disordium salt. The composition may also comprise an additional tooth whitening agent. Methods of whitening the teeth are also described.

This invention relates to dental compositions and methods, and in particular to compositions and methods for enhancing or improving the whiteness of teeth.

Staining or discolouration of teeth can arise as a result of a number of factors, including

1) exposure of the teeth to staining agents such as tea, coffee, smoke etc, which can cause entrapment or attachment of stain molecules to the tooth surface;

2) ingestion of certain materials, eg certain drugs such as tetracycline, during tooth formation, which can cause discolouration of the tooth enamel;

3) trauma to the tooth that can cause leakage of blood breakdown products into the enamel and thus discolouration.

A number of different methods for tooth whitening are known. These include:

1 ) Physical abrading of the stain to remove it. This involves the use of harsher abrasives than are found in typical tooth-whitening toothpaste formulations. Brushing or a similar polishing or scrubbing action is required to complement the action. This approach suffers from the disadvantage that it may cause excessive abrasion to the tooth enamel.

2) Oxidising or bleaching agents such as peroxides are widely used to effect removal of both extrinsic and intrinsic stains. Such agents can be applied either as pastes or gels. However, the treatment may require considerable time to achieve good results. The concentration of oxidising agent may be increased to reduce the time required to achieve effective results, but this may lead to safety concerns.

3) Enzymatic systems comprising proteases such as papain that are claimed to remove stain by dissolution of the pellicle entrapping the stain.

4) Chelating agents have also been used to whiten teeth by interacting either with inorganic components of the tooth and tartar or with stain molecules. Typical examples of such agents are pyrophosphates and polyphosphates.

The natural colour of the tooth is very rarely pure white, mainly because the dentin, which contains a substantial amount of collagen, is yellowish and the enamel above it is not completely opaque. The “whitening” approaches 3) and 4) described above are primarily based on interactions with acquired enamel pellicle, plaque and dental calculus. The mechanism employed is to either remove or weaken these tooth surface adsorbents and precipitates. Approach 1) simply polishes the enamel using abrasives and thus removes stains. However, selection of abrasives always involves a balance between its efficacy and mechanical destruction of the enamel. The use of bleaching agents, as in approach 2), involves chemical removal of the colour of organic molecules on or within the tooth surface and thus achieves a better cosmetic whitening effect.

Fluorescent whitening agents have been known for many years. Most molecules categorised as fluorescent whitening agents contain chromophores which contain conjugated double bonds that absorb UV light. By re-emitting the absorbed light at higher wavelength regions, within the visible spectrum, the fluorescent whitening agents can alter the visual appearance of a substrate to which they are applied. The optical properties of the chromophores can be varied by electron donors or acceptors. Hitherto, however, the use of such compounds has been confined to industrial fields such as the textile, paper and detergent industries.

It has now been surprisingly found that fluorescent whitening agents can advantageously be incorporated in dental compositions, and can be used in methods for improving or enhancing the whiteness of teeth. The present invention thus provides dental compositions and methods that overcome or substantially mitigate the disadvantages of the prior art approaches described above.

According to a first aspect of the invention, there is provided a dental composition comprising a fluorescent whitening agent and a dentally acceptable diluent or carrier.

The composition according to the invention is advantageous primarily in that it is found that the composition, containing the fluorescent whitening agent, is effective in improving the whiteness of teeth to which it is applied. Such an effect is believed to be due to an adherence of the fluorescent whitening agent to the teeth, presumably to the pellicle, the outer layer of proteins and carbohydrates that coats the tooth enamel. Such adherence, if indeed it does occur, could not have been predicted from the known industrial applications of fluorescent whitening agents.

As used herein, a “dental composition” is any composition suitable and intended for administration to the buccal cavity for the purpose of improving the appearance of the teeth. In the context of this invention, this means improving or enhancing the whiteness of the teeth. The dental composition may, for instance, be formulated as a toothpaste, mouthrinse, toothgel, tooth paint or a dental gel. By a “fluorescent whitening agent” is meant a compound that absorbs invisible ultra-violet light and which re-emits the light in the visible spectral range. The compound thus absorbs light of wavelength less than, say, 380 nm, and re-emits light in the wavelength range 380 nm(violet) to 780 nm (red). To achieve a whitening effect, the re-emitted light should complement the light reflected from the substrate to which the fluorescent whitening agent is applied, so that a more uniform spectral distribution is achieved across the whole visible range. It is particularly preferred, in order to compensate for the yellow cast of discoloured teeth, that the fluorescent whitening agent should emit in the blue region of the visible spectrum, eg in the range 400 nm to 450 nm.

The fluorescent whitening agent may be any compound that exhibits appropriate spectroscopic properties, and which is acceptable for administration in the manner contemplated by this invention, eg from the point of view of its toxicological properties.

Fluorescent whitening agents of potential utility in the present invention include the following classes of compound:

a) bis-styrylbiphenyl compounds, ie compounds having the following chromophore system:

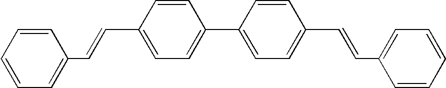

b) derivatives of stilbene, ie compounds having the following chromophore system: 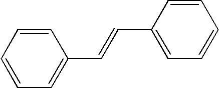
and in particular bis-triazineamine derivatives of such compounds having the following chemical backbone:

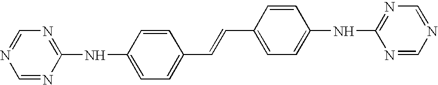

c) coumarins having the following chromophore system:

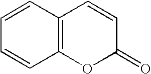

d) naphthalimide derivatives having the following chromophore system:

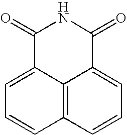

e) benzoxazole derivatives having the following chromophore system: 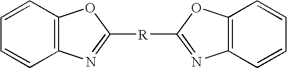
in which R represents a conjugated group, eg an ethylene group or a stilbene moiety (see above);

f) pyrazoline and bis-pyrazoline derivatives having the following chromophore systems:

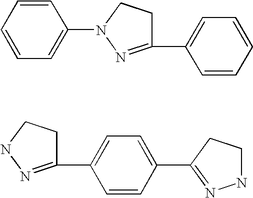

It will be appreciated that all of the chromophore systems defined above may carry one or more substituent atoms or groups, and a great number of derivatives can be made by chemical substitution.

Preferred bis-styrylbiphenyl compounds of utility in the invention may be represented by the general formula: 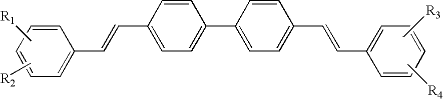
in which R₁ is —SO₃M and R₂, R₃ and R₄, which may be the same or different, are selected from R₅, —SO₃M, halogen (particularly Cl), —CN, —OC(═O)R₅, —COOR₅, —SO₂N(R₅)₂ and —CON(R₅)₂, wherein R₅ represents hydrogen or C₁₋₈ alkyl and M represents hydrogen or a Group I metal, eg Na, K or Li. Particularly preferred compounds are those in which R₃ is the same as R₁, and R₂ and R₄ are the same and are selected from R₅, halogen, —CN, —OC(═O)R₅, —COOR₅, —SO₂N(R₅)₂ and —CON(R₅)₂.

Examples of bis-styrylbiphenyl compounds that may be used are 4,4′-bis(2-sulfostyryl)biphenyl (sold under the name of Tinopal CBS) and disodium 4,4′-bis(3-sulfo-4-chlorostyryl)biphenyl, both of which are supplied by Ciba Speciality Chemicals pic.

Examples of bistriazineamino derivatives of stilbene are disodium 4,4′-bis[(4-anilino-6-morpholino-1,3,5-triazin-2-yl)amino]stilbene-2,2′-disulfonate (sold under the trade name Tinopal DMS), disodium 4,4′-bis{[4-anilino-6-(N-methyl-N-2-hydroxyethyl)amino-1,3,5-triazin-2-yl]amino}stilbene-2,2′-disulfonate, and disodium 4,4′-bis[(4-anilino-6-methylamino-1,3,5-triazin-2-yl)aminolstilbene-2,2′-disulfonate (all of which are supplied by Ciba Specialty Chemicals pic).

An example of a coumarin fluorescent whitening agent is Coumarin 102, the structure of which is:

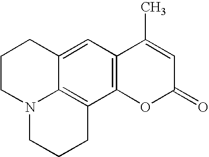

An example of a naphthalimide fluorescent whitening agent is 1,8-naphthalimide, the structure of which is:

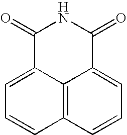

A particularly preferred fluorescent whitening agent is 4,4′-bis(2-sulfostyryl)biphenyl or a salt or other soluble derivative thereof, eg the disodium salt, one such compound being sold under the trade name Tinopal CBS-X by Ciba Speciality Chemicals.

The invention may involve the use of a compound known for use as a fluorescent whitening agent in other fields of application (eg for application to textile or for use in a detergent composition), or the use of a compound having the required optical properties which is either a novel compound or a compound which, while known per se, has not previously been used as a fluorescent whitening agent.

The content of fluorescent whitening agents in the composition according to the invention will depend on a number of factors, including the particular fluorescent whitening agent used and the magnitude of the desired whitening effect. A distinction may also be drawn between compositions intended for clinical use by a dental surgeon, and those for self-administration (eg at home) by the patient. In the former case, the concentration of fluorescent whitening agent may be up to 1000 ppm, but is more commonly 500 ppm or less, say between 50 ppm and 500 ppm. In the latter case, the concentration of fluorescent whitening agent will generally be much lower, say up to 100 ppm, and more usually up to 50 ppm, eg between 5 ppm and 50 ppm.

According to another aspect of the invention, there is provided a method for improving or enhancing the appearance of the teeth, in particular for whitening of the teeth, which method comprises application to the teeth of a dental composition comprising a fluorescent whitening agent and a dentally acceptable diluent or carrier.

To achieve maximal results, the treatment will normally be repeated at intervals for a period of several weeks or months. For instance, the treatment may be applied initially by a dental surgeon, who may also provide one or more subsequent treatments at intervals of, say, one or two days, or one or two weeks. Between these clinical applications of the composition, the patient may himself apply regular treatments, eg once or several times each day.

In preferred embodiments, the method according to the invention involves treatment of the teeth, in addition to treatment with the fluorescent whitening agent, with one or more additional tooth whitening agents. Such other agents may, for instance, be

a) abrasive agents effective in physically removing stains from the tooth enamel;

b) oxidising or bleaching agents such as peroxides;

c) enzymatic systems, eg those including proteases such as papain; and

d) chelating agents, eg pyrophosphates and polyphosphates.

Thus, according to more specific aspects of the invention, there are provided

a) a method for improving or enhancing the appearance of the teeth, in particular for whitening of the teeth, which method comprises application to the teeth of an additional tooth whitening agent and a fluorescent whitening agent; and

b) a dental composition comprising an additional tooth whitening agent and a fluorescent whitening agent, together with a dentally acceptable diluent or carrier.

The effects of the additional whitening agent and the fluorescent whitening agent may be more than simply cumulative. It is believed that, at least in some instances, treatment of the teeth with the additional whitening agent may enhance the binding to the teeth of the fluorescent whitening agent, and thereby lead to a greater-than-additive overall effect of the two agents. In addition, treatment with the additional whitening agent may lead to greater uniformity of the enhanced whiteness achieved by application of the fluorescent whitening agent.

The additional whitening agent may be any agent effective to improve the whiteness of the teeth. The preferred form of additional whitening agent, however, is an oxidising or bleaching agent.

Suitable forms of bleaching agent include compounds containing (or compounds capable, in use, of generating) peroxide or chlorite ions. Examples of peroxide or peroxide-generating compounds include hydrogen peroxide, sodium percarbonate, sodium perborate, sodium persilicate, sodium perphosphate, calcium peroxide and sodium peroxide. The presently most preferred peroxide bleaching agents are hydrogen peroxide or adducts such as urea peroxide (carbamide peroxide).

Examples of chlorite bleaching agents include alkali metal chlorites such as lithium chlorite, potassium chlorite and, especially, sodium chlorite, and alkaline earth metal chlorites such as calcium chlorite, magnesium chlorite and barium chlorite. Chlorite salts of other metals, and other salts, eg polymeric salts, may also be used. Water-soluble chlorites are generally preferred. Such compounds may generate chlorine dioxide upon contact with an acid.

Particularly preferred bleaching agents for use in the invention are hydrogen peroxide and urea peroxide (carbamide peroxide).

The concentration of additional whitening agent used in the method according to the invention will depend on factors such as the nature of the additional whitening agent and the magnitude of the desired whitening effect. Again, a distinction can be drawn between clinical and home use. In the case of the preferred bleaching agent hydrogen peroxide, the bleaching agent will typically be present in the composition at a level sufficient to generate a concentration of peroxide of up to 50% by weight in the case of a composition for clinical use, more typically up to 30%, and usually between 1% and 15%, typically from 5% to 15%. In compositions for home use, on the other hand, the concentration of bleaching agent will generally be much lower, usually less than 10%, and often less than 1%, eg 0.1%.

Application of the fluorescent whitening agent and the additional whitening agent may be simultaneous, in which case the fluorescent whitening agent and the additional whitening agent may be formulated in the same composition.

Alternatively, the additional whitening agent and the fluorescent whitening agent may be applied sequentially, in which case they will be formulated in separate compositions. In such a case, the additional whitening agent is applied first, followed by the fluorescent whitening agent.

The method according to the invention may be a clinical method carried out by, or under the direct supervision of a professional dental surgeon. Alternatively, the method may be carried out by a user at home, the composition of the invention being self-administered. The method may involve both types of treatment, these being an initial professional treatment, and optionally one or more subsequent professional treatments, and subsequent maintenance treatment performed by the user.

In a presently favoured treatment regime, the fluorescent whitening agent and the additional tooth whitening agent are applied separately in a first stage of the tooth whitening process, and then applied together in a second stage of the process. The first stage may be carried out by a dental surgeon, and the second stage may be carried out by the patient.

Application of the compositions used in the invention to the teeth may involve simply bringing the composition into contact with the teeth, eg by introducing the composition into the buccal cavity and allowing it to disperse within that cavity. Toothguards or the like may be used to constrain the composition to the vicinity of the teeth.

In general, the composition may be applied to the teeth for a period of from several minutes, say from 1 minute to 5 minutes, up to one hour or so. Afterwards, the composition is preferably removed from the buccal cavity by rinsing.

Treatment by a dental surgeon will normally begin with a conventional scale-and-polish treatment. This may be followed by one or more applications of a bleaching agent, eg at daily or weekly intervals, in a conventional manner. The fluorescent whitening agent may then be applied after the last bleaching treatment.

Maintenance treatment may then be performed by the patient, eg by regular use of a toothpaste containing relatively low concentrations of fluorescent whitening agent and optionally a bleaching agent or other tooth whitening agent. Further clinical treatments may then be required, eg at six or twelve month intervals, to maintain the whiteness of the teeth.

The compositions according to the invention may, as appropriate, contain further components which are well known to those skilled in the art of dental preparations such as, for example, humectants, surfactants, abrasives, fluoride sources, desensitising agents, flavourings, colourings, sweeteners, antimicrobial agents to act as preservatives, bactericides and/or anti-plaque agents, anti-calculus (anti-tartar) agents, structuring agents, chelating agents, whitening agents, vitamins and any other therapeutic actives. The choice and amount of additives for any particular form of composition will be evident to, or readily determined by, those skilled in the art.

Suitable humectants for use in dentifrice compositions include polyhydric alcohols such as xylitol, sorbitol, glycerol, propylene glycol and polyethylene glycols. Sorbitol, and mixtures of glycerol and sorbitol or sorbitol and xylitol, are particularly effective. A humectant helps to prevent dentifrice compositions from hardening on exposure to air, and may also provide a moist feel, smooth texture, flowability, and a desirable sweetness in the mouth. Suitably, such humectants may comprise from about 0-85%, preferably from about 20-80% by weight of the composition. Suitable surfactants for use in dentifrices, mouthwashes etc. are usually water-soluble organic compounds, and may be anionic, non-ionic, cationic or amphoteric species.

Anionic surfactants include the water-soluble salts of C10-C18 alkyl sulphates (eg sodium lauryl sulfates), water soluble salts of C10-C18 ethoxylated alkyl sulphates, water soluble salts of C10-C18 alkyl sarcosinates, the water-soluble salts of sulfonated monoglycerides of C10-C18 fatty acids (eg sodium coconut monoglyceride sulfonates), alkyl aryl sulfonates (eg sodium dodecyl benzene sulfonate), sodium salts of the coconut fatty acid amide of N-methyltaurine and sodium salts of long chain olefin sulfonates (eg sodium C14-C16 olefin sulfonates).

Non-ionic surfactants suitable for use in oral compositions include the products of the condensation of alkylene oxide groups with aliphatic or alkylaromatic species, and may be for example, polyethylene oxide condensates of alkyl phenols, ethylene oxide/propylene oxide copolymers (available from BASF Wyandotte Chemical Corporation under the trade name ‘Pluronic’), ethylene oxide/ethylene diamine copolymers, ethylene oxide condensates of aliphatic alcohols, long chain tertiary amine oxides, long chain tertiary phosphine oxides, long chain dialkyl sulfoxides and mixtures thereof. Alternatives include ethoxylated sorbitan esters such as those available from ICI under the trade name “Tween”.

Cationic surfactants are generally quaternary ammonium compounds having one C8-C18 alkyl chain and include, for example, lauryl trimethylammonium chloride, cetyl trimethylammonium bromide, cetyl pyridinium chloride, diisobutylphenoxyethoxyethyidimethylbenzylammonium chloride, coconut alkyl trimethylammonium nitrite and cetyl pyridinium fluoride.

Also useful are benzyl ammonium chloride, benzyl dimethyl stearylammonium chloride, and tertiary amines having one C1-C18 hydrocarbon group and two (poly)oxyethylene groups.

Amphoteric surfactants may be aliphatic secondary and tertiary amines comprising aliphatic species which may be branched or unbranched, and in which one of the aliphatic species is a C8-C18 species and the other contains an anionic hydrophilic group, for example, sulfonate, carboxylate, sulfate, phosphonate or phosphate. Examples of quaternary ammonium compounds are the quaternized imidazole derivatives available under the trade name ‘Miranol’ from the Miranol Chemical Company. Other amphoteric surfactants that may be employed are fatty acid amido alkyl betaines where one alkyl group is commonly C10-C12 such as cocamido propyl betaine, for example Tego Betain supplied by T H Goldschmitt.

Flavouring agents may be added to increase palatability and may include, for example, menthol, oils of peppermint, spearmint, wintergreen, sassafras and clove. Sweetening agents may also be used, and these include D-tryptophan, saccharin, dextrose, aspartame, levulose, acesulfam, dihydrochalcones and sodium cyclamate.

Colouring agents and pigments may be added to improve the visual appeal of the composition. Suitable colourants include dyes and pigments. A suitable and commonly used pigment is pigment grade titanium dioxide, which provides a strong white colour.

Suitably, as described above, the compositions of the invention may include a further antimicrobial agent as a preservative, antibacterial and/or anti-plaque agent. Suitable antimicrobial agents include water soluble sources of certain metal ions such as zinc, copper and silver such as zinc citrate and silver chloride, cetyl pyridinium chloride, the bis-biguanides (such as chlorhexidine), aliphatic amines, phenolics such as bromochlorophene and triclosan, salicylanilides and quaternary ammonium compounds. Optionally, the formulations may also contain enzymes that will disrupt the pellicle or interfere with bacterial intercellular polysaccharides. Examples include proteases such as papain and bromelain or dextranases. Natural enzymatic biocidal systems such as a system comprising lactoperoxidase and glucose oxidase may also be employed.

The composition may also comprise an anti-calculus (anti-tartar) agent. Suitable anti-calculus agents include zinc salts such as zinc citrate and zinc chloride, polyphosphates and pyrophosphates. Suitable pyrophosphates include the sodium and potassium pyrophosphates, preferably disodium pyrophosphate, dipotassium pyrophosphate, tetrasodium pyrophosphate and tetrapotassium pyrophosphate. A preferred source of pyrophosphate is a mixture of tetrasodium pyrophosphate and tetrapotassium pyrophosphate. Suitable polyphosphates include sodium tripolyphosphate.

Structuring (gelling) agents may be required in, for example, dentifrices and gums to provide desirable textural properties and “mouthfeel”. Suitable agents include natural gum binders such as gum tragacanth, xanthan gum, gum karaya and gum arabic, seaweed derivatives such as Irish moss and alginates, smectite clays such as bentonite or hectorite, carboxyvinyl polymers and water-soluble cellulose derivatives such as hydroxyethyl cellulose and sodium carboxymethyl cellulose. Improved texture may also be achieved, for example, by including colloidal magnesium aluminium silicate.

Suitable vitamins for inclusion in the dental preparations of the present invention include vitamins A, B5, B6, C and E.

The composition may also comprise a polymer to enhance delivery and retention of the active ingredients to the tooth surface. Suitable polymers may include PVM/MA (poly(vinylmethylether/maleic acid)) copolymers eg Gantrez S-97 from ISP, PVP (polyvinylpyrrolidone) or PVPNA (polyvinylpyrrolidone/vinyl acetate) copolymers, eg Plasdone S-630 from ISP, and similar materials from other suppliers.

Where the composition contains any of the further ingredients or excipients mentioned above, the concentrations of such ingredients may be generally similar to those conventional in dental compositions of the corresponding form.

The invention is illustrated by the following non-limiting Examples, in which

a) Examples 1 to 3 are examples of compositions containing a relatively low concentration of fluorescent whitening agent, suitable for home use;

b) Examples 4 to 6 are examples of compositions containing a relatively high concentration of a fluorescent whitening agent and, in the case of Example 6, a bleaching agent, these compositions being suitable for professional use; and

c) Example 7 is a toothpaste formulation suitable for home use, containing both fluorescent whitening agent and bleaching agent.

In all cases, the formulations can generally be prepared by simple admixture of the various ingredients, in a manner that will be evident to, or readily determined by, those skilled in the art.

### EXAMPLE 1

Tooth Gel

| Material | % w/w |
|---|---|
| Sorbitol 70% solution | 68.00 |
| PEG 1500 | 1.39 |
| Sodium hydroxide | 0.05 |
| Sodium monofluorophosphate | 0.80 |
| Sodium saccharin | 0.26 |
| Silica abrasive | 6.94 |
| Silica Thickener | 9.17 |
| SLS Needles | 1.00 |
| Flavouring | 1.00 |
| Sodium carboxymethylcellulose | 1.00 |
| Tinopal CBS-X | 0.005 |
| Purified water | q.s. |

### EXAMPLE 2

Toothpaste

| Material | % w/w |
|---|---|
| Sorbitol 70% solution | 50.00 |
| PEG 1500 | 1.39 |
| Sodium hydroxide | 0.05 |
| Sodium fluoride | 0.22 |
| Sodium saccharin | 0.26 |
| Silica abrasive | 6.94 |
| Silica Thickener | 9.17 |
| SLS Needles | 1.00 |
| Flavouring | 1.00 |
| Sodium carboxymethylcellulose | 1.00 |
| Tinopal CBS-X | 0.005 |
| Titanium dioxide | 0.50 |
| Purified water | q.s. |

### EXAMPLE 3

Mouthwash

| Ingredients | % w/w |
|---|---|
| Sorbitol 70% solution | 10.00 |
| Sodium Fluoride | 0.05 |
| Sodium Saccharin | 0.03 |
| Cetyl Pyridinium Chloride | 0.05 |
| Ethanol 96% | 7.00 |
| Flavouring | 0.15 |
| Tween 20 | 0.30 |
| Tinopal CBS-X | 0.005 |
| Purified Water | q.s. |

### EXAMPLE 4

Gel

| Ingredients | % w/w |
|---|---|
| Pluronic F-127 | 22.00 |
| Glycerin | 10.00 |
| Sodium Saccharin | 0.26 |
| Flavouring | 1.00 |
| Tinopal CBS-X | 0.05 |
| Purified Water | q.s. |

### EXAMPLE 5

Toothpaint

| Ingredients | % w/w |
|---|---|
| Pluronic F-127 | 5.00 |
| Glycerin | 10.00 |
| Ethanol 96% | 30.00 |
| Tinopal CBS-X | 0.05 |
| Purified Water | q.s. |

### EXAMPLE 6

Gel with Bleach and FWA

| Ingredients | % w/w |
|---|---|
| Pluronic F-127 | 25.00 |
| Glycerin | 35.00 |
| Carbamide Peroxide | 15.00 |
| Tinopal CBS-X | 0.05 |
| Sodium Saccharin | 0.26 |
| Flavouring | 1.00 |
| Purified Water | q.s. |

### EXAMPLE 7

Toothpaste with Bleach and FWA

| Ingredients | % w/w |
|---|---|
| Glycerin | 35.00 |
| Sodium Saccharin | 0.26 |
| Sodium Fluoride | 0.22 |
| Silica Abrasive | 6.67 |
| Silica Thickener | 10.83 |
| SCMC | 1.00 |
| Titanium Dioxide | 0.50 |
| SLS | 1.50 |
| PEG 1500 | 1.39 |
| Flavouring | 1.00 |
| Carbamide Peroxide | 0.30 |
| Tinopal CBS-X | 0.005 |
| Purified Water | q.s. |

The efficacy of the method of the invention (employing Tinopal CBS-X, referred to as FWA, as fluorescent whitening agent and hydrogen peroxide as bleaching agent) is demonstrated by the following measurements.

Methods

1) Treatment of Teeth

Extracted caries-free teeth collected from dental hospitals were briefly brushed to remove any skin tissue attached. The teeth were then sterilized by autoclaving at 121° C. for 25 mins. All teeth were stored in an airtight plastic container containing a solution of Phosphate Buffered Saline (PBS) with a few crystals of thymol as an antibacterial agent until required for use. Twenty four hours before the study, each tooth was glued onto a numbered glass microscope slide. A pellicle was formed on the teeth by soaking in sterilised artificial saliva with the composition set out in Table 1.

After the 24 hr period of pellicle formation, the teeth were then brushed with an Oral B Plaque remover electric toothbrush for 2 mins, with a standard fluoride toothpaste, the composition of which is set out in Table 2:

Excess toothpaste was removed by briefly rinsing each tooth with deionised water. The teeth were returned to a fresh aliquot of artificial saliva and then measured. The measured whiteness index (determined by image analysis as described below) was taken as the baseline against which subsequent measurements were compared.

The teeth were bleached by immersion in a 15% hydrogen peroxide solution (prepared from 45g Urea Hydrogen Peroxide in 55g deionised water) for 1 hr. Teeth were returned to a fresh aliquot of artificial saliva and a Post-Bleach measurement taken.

FWA was applied to the teeth by immersion in a 500 ppm FWA solution (50 mg FWA in 100 ml deionised water stirred for 30 minutes) for 1 hr. The teeth were returned to a fresh aliquot of artificial saliva and a post-FWA measurement taken.

2) Image Analysis

Each tooth was imaged using a Kodak DCS 410 camera, which was mounted 10 cm above and perpendicular to the specimen, using aperture F11 and a shutter speed of 1/10 second. The standardised lighting source comprised two UV tubes and two 6500K ‘daylight’ tubes. Each image was transferred to Adobe PhotoShop software. For each image, an 8 mm² area was analysed and an average Adobe L,a,b reading obtained using the software. All L,a,b values were converted to CIE L*,a*,b* and then to the CIE whiteness index W, using the equation:
W=Y+800(xₙ−x)+1700(yₙ−y)
where Y is the Y-tristimulus value of the specimen, x and y, and xₙ and yₙ are the chromaticity coordinates for the specimen and for the perfect diffuser for the CIE 1931 2° Standard Observer, respectively.
Results

Six groups of three teeth were studied. For each group of teeth, treatment with 15% H₂O₂ produced an increase in whiteness index, but further treatment with FWA produced a further increase in whiteness. For the whole group of teeth studied, the average percentage increase in whiteness index was 17% for the H₂O₂ alone, and 24% for the combined treatment using H₂O₂ and FWA.

Measurements Using Other FWAs

Similar measurements to those described above were made using the following commercially-available FWAs in place of the Tinopal CBS-X:

1,8 Naphthalimide (Sigma Aldrich)

Blankophor CLE (stilbene derivative—Bayer Speciality Products)

Coumarin 102 (Sigma Aldrich)

Tinopal 5BM-GX (stilbene derivative—Ciba)

Tinopal AMS-GX (stilbene derivative—Ciba)

Tinopal LMS-X (stilbene derivative—Ciba)

In each case, a measurable increase in whiteness index was observed compared with treatment with H₂O₂ alone.

Measurements Using Alternative Bleaching Agent

Similar measurements were made using sodium chlorite (generating chlorine dioxide in use) as bleaching agent in place of H₂O₂, and Tinopal CBS-X as FWA. For the group of teeth studied, the average percentage increase in whiteness index was 8% for the bleach alone, and 41% for the combined treatment using chlorine dioxide and FWA.

TABLE 1
Formula for Artificial Saliva

| Material | Concentration | |
|---|---|---|
| Calcium Chloride | 2.0 | mM |
| Magnesium Chloride | 4.2 | mM |
| Sodium Chloride | 4.0 | mM |
| Sodium Thiocyanate | 1.3 | mM |
| Sodium Fluoride | 15 | μM |
| Potassium Iodide | 8.0 | nM |
| Potassium Bicarbonate | 5.0 | mM |
| Potassium Dihydrogen Phosphate | 6.0 | mM |
| Potassium Chloride | 10.0 | mM |
| Urea | 3.2 | mM |
| Glucose | 0.55 | mM |
| Lactoferrin | 5.4 | mg/l |
| Lysozyme | 0.264 | g/l |
| α-Amylase | 0.38 | g/l |
| Lactoperoxidase | 2.0 | mg/l |
| Serum Albumin | 2.2 | g/l |

TABLE 2
Composition of standard toothpaste

| Material | % w/w |
|---|---|
| Sorbitol 70% soln | 25.00 |
| Sodium Saccharin | 0.26 |
| Sodium Fluoride | 0.22 |
| Silica Abrasive (Medium) | 6.67 |
| Silica Thickener | 10.83 |
| Sodium Carboxymethyl Cellulose | 1.50 |
| Titanium Dioxide | 0.50 |
| Sodium Lauryl Sulphate | 1.50 |
| Polyethylene Glycol 1500 | 1.39 |
| Sodium Bicarbonate | 1.00 |
| Flavour | 1.00 |
| Sodium Benzoate | 0.50 |
| Purified Water | To 100% |

1-32. (canceled)

33. A method for improving or enhancing the appearance of the teeth, in particular for whitening of the teeth, which method comprises application to the teeth of a dental composition comprising a fluorescent whitening agent selected from derivatives of stilbene having the following chromophore system: 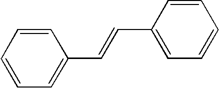
as the sole tooth whitening agent in the composition, or with one or more additional tooth whitening agents selected from
a) abrasive agents effective in physically removing stains from the tooth enamel;
b) chlorite oxidising or bleaching agents;
c) enzymatic systems; and
d) chelating agents;
and a dentally acceptable diluent or carrier.

34. A method for improving or enhancing the appearance of the teeth, in particular for whitening of the teeth, which method comprises application to the teeth of a dental composition comprising a fluorescent whitening agent selected from bis-styrylbiphenyl compounds having the following chromophore system: 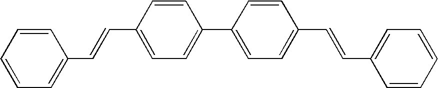
and a dentally acceptable diluent or carrier, optionally with an additional tooth whitening agent.

35. A method as claimed in claim 33, wherein the composition is formulated as a toothpaste, mouthrinse, toothgel, tooth paint or dental gel.

36. A method as claimed in claim 33, wherein the fluorescent whitening agent absorbs light of wavelength less than 380 nm and re-emits light in the wavelength range 400 nm to 450 nm.

37. A method as claimed in claim 33, wherein the fluorescent whitening agent is selected from bis-triazineamine derivatives of compounds having the following chemical backbone:

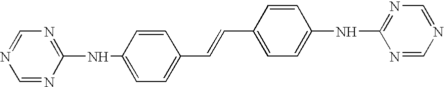

38. A method as claimed in claim 33, wherein the fluorescent whitening agent is selected from the group consisting of disodium 4,4′-bis[(4-anilino-6-morphoiino-1,3,5-triazin-2-yl)amino]stilbene-2,2′-disulfonate, disodium 4,4′-bis{[4-anilino-6-(N-methyl-N-2-hydroxyethyl)amino-1,3,5-triazin-2-yl]amino}stilbene-2,2′-disulfonate, and disodium 4,4′-bis[(4-anilino-6-methylamino-1,3,5-triazin-2-yl)aminolstilbene-2,2′-disulfonate.

39. A method as claimed in claim 34, wherein the fluorescent whitening agent is a bis-styrylbiphenyl compound of the general formula: 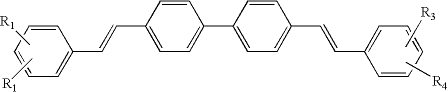
in which R₁ is —SO₃M and R₂, R₃ and R₄, which may be the same or different, are selected from R₅, —SO₃M, halogen (particularly Cl), —CN, —OC(═O)R₅ᵢ-000R₅, —SO₂N(R₅)₂ and —CON(R₅)₂, wherein R₅ represents hydrogen or C₁₋₈ alkyl and M represents hydrogen or a Group I metal, eg Na, K or Li.

40. A method as claimed in claim 39, wherein R₃ is the same as R1, and R₂ and R₄ are the same and are selected from R₅, halogen, —CN, —OC(50 O)R₅, -000R₅, —SO₂N(R₅)₂ and —CON(R₅)₂.

41. A method as claimed in claim 40, wherein the fluorescent whitening agent is 4,4′-bis(2-sulfostyryl)biphenyl or a salt or other soluble derivative thereof.

42. A method as claimed in claim 41, wherein the fluorescent whitening agent is disodium 4,4′-bis(2-sulfostyryl)biphenyl.

43. A method as claimed in claim 33, wherein the concentration of fluorescent whitening agent in the composition is less than 1,000 ppm.

44. A method as claimed in claim 43, wherein the concentration of fluorescent whitening agent in the composition is in the range 50 ppm to 500 ppm.

45. A method as claimed in claim 43, wherein the concentration of fluorescent whitening agent in the composition is less than 100 ppm.

46. A method as claimed in claim 45, wherein the concentration of fluorescent whitening agent in the composition is in the range 5 ppm to 50 ppm.

47. A method as claimed in claim 33, wherein the method further comprises the application of an additional tooth whitening agent.

48. A method as claimed in claim 47, wherein application of the additional tooth whitening agent is simultaneous with application of the fluorescent whitening agent.

49. A method as claimed in claim 47, wherein the composition comprises an additional tooth whitening agent.

50. A method as claimed in claim 34, wherein the composition comprises a bleaching agent as an additional tooth whitening agent.

51. A method as claimed in claim 50, wherein the bleaching agent is a peroxide.

52. A method as claimed in claim 51, wherein the peroxide is hydrogen peroxide or a compound that generates hydrogen peroxide in use.

53. A method as claimed in claim 50, wherein the bleaching agent is a chlorite bleaching agent.

54. A method for improving or enhancing the appearance of the teeth, in particular for whitening of the teeth, which method comprises application to the teeth of a dental composition comprising a fluorescent whitening agent selected from the group consisting of derivatives of stilbene having the following chromophore system: 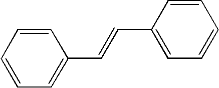
and an additional tooth whitening agent wherein the method comprises the sequential application to the teeth of the additional tooth whitening agent followed by the fluorescent whitening agent.

55. A method as claimed in claim 54, wherein the fluorescent whitening agent compounds have the following chromophore system:

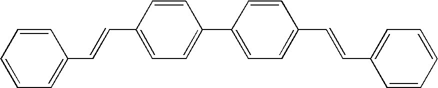

56. A method as claimed in claim 54, wherein the additional tooth whitening agent is a bleaching agent.

57. A method as claimed in claim 56 wherein the bleaching agent is a peroxide.

58. A method as claimed in claim 57, wherein the peroxide is hydrogen peroxide or a compound that generates hydrogen peroxide in use.

59. A method as claimed in claim 54, wherein one or more applications of the additional tooth whitening agent precede application of the fluorescent whitening agent.

60. A method as claimed in claim 54, wherein the fluorescent whitening agent is selected from the group consisting of disodium 4,4′-bis(2-sulfostyryl)biphenyl, 4,4′-bis(2-sulfostyryl)biphenyl, disodium 4,4′-bis(3-sulfo-4-chlorostyryl)biphenyl, disodium 4,4′-bis[(4-anilino-6-morpholino-1,3,5-triazin-2-yl)aminoistilbene-2,2′-disulfonate, disodium 4,4′-bis{[4-anilino-6-(N-methyl-N-2-hydroxyethyl)amino-1,3,5-triazin-2-yll amino)stilbene-2,2′-disulfonate, and disodium 4,4′-bis[(4-anilino-6-methylamino-1,3,5-triazin-2-yl)aminolstilbene-2,2′-disulfonate.

61. A method as claimed in claim 54, comprising a first stage in which the additional tooth whitening agent is applied by a dental surgeon, and a second stage in which the fluorescent whitening agent and the additional tooth whitening agent are applied, simultaneously or sequentially, by the patient.

62. A method as claimed in claim 61, wherein in the first stage the additional tooth whitening agent is applied first, followed by a fluorescent whitening agent.

63. A method as claimed in claim 60, wherein in the second stage, the additional tooth whitening agent and the fluorescent whitening agent are applied simultaneously.
